(19) United States
(12) Patent Application Publication
(10) Pub. No.: US 2019/0022147 A1
(43) Pub. Date: Jan. 24, 2019

(54) Treatment of Glioma by Amniotic Fluid Stem Cells and Exosomes Derived Thereof

(71) Applicant: Creative Medical Technologies, Inc, Phoenix, AZ (US)

(72) Inventors: Thomas Ichim, San Diego, CA (US); Amit Patel, Salt Lake City, UT (US); Santosh Kesari, Santa Monica, CA (US)

(73) Assignee: Creative Medical Technologies, Inc, Phoinex, AZ (US)

(21) Appl. No.: 16/044,256

(22) Filed: Jul. 24, 2018

Related U.S. Application Data
(60) Provisional application No. 62/536,447, filed on Jul. 24, 2017.

(51) Int. Cl. A61K 35/50; A61P 35/00; C12N 5/073

(52) U.S. Cl. CPC A61K 35/50; A61P 35/00; C12N 5/0605; C12N 2501/999; C12N 2501/13; C12N 2501/30; C12N 2501/73; C12N 2500/02

(57) ABSTRACT

Disclosed are compositions of matter, therapeutic protocols, and cellular reprogramming means to inhibit glioma or other brain neoplasia. In one embodiment the invention provides administration of amniotic fluid derived stem cells at concentrations of 1 million to 200 million administered in a manner to provide a differentiation stimulation, resulting in reduction of malignant potential. In other embodiments, an unexpected synergy of cancer inhibitory soluble factor production is disclosed by combined cultures between amniotic fluid stem cells and monocytes. In all embodiments cells may be autologous or allogeneic. The invention provides means of augmenting efficacy of immunotherapy, chemotherapy, and radiotherapy.

### CROSS-REFERENCE TO RELATED APPLICATIONS

This application claims priority to U.S. Provisional Application No. 62/536,447, filed Jul. 24, 2017, which is incorporated herein by reference in its entirety

### FIELD OF THE INVENTION

The invention pertains to the disease glioma. More particularly, the invention relates to new methods of treating glioma by extracting and expanding cells from amniotic fluid and administering said cells into patients suffering from glioma.

### BACKGROUND

Glioblastoma multiforme is the most common and most aggressive form of primary brain tumour with an incidence of 2.8 cases per 100,000 per year in the United States. Due to the highly infiltrative nature of GBM and the intrinsic chemoresistance of GBM cells, 80% of tumours recur within 2 cm of the tumour resection cavity or in the context of tumours treated by radiotherapy and chemotherapy alone, recurrence most commonly occurs adjacent to the original tumour mass. As systemic dissemination of GBM is extremely rare and the median survival for recurrent GBM is typically less than 1 year, there is a clear and rational need for effective strategies aimed at improving local tumour control.

Techniques attempted in clinical trials to improve the local control of GBM have included the direct infusion or implantation of conventional chemotherapeutic agents such as carmustine, paclitaxel and topotecan, or novel cytotoxic agents, including oncolytic herpes simplex and adenoviral vector viral and non-viral mediated gene therapy and immunotoxins such as IL13-PE38QQR, into the tumour mass, resection cavity or peritumoural tissue. To date, the only technique of localised drug delivery that has become clinically accepted is the implantation of carmustine wafers (Gliadel) into the tumour resection cavity. However, a recent Cochrane Collaboration Review of the use of Gliadel wafers concluded that in combination with radiotherapy, Gliadel has survival benefits in the management of primary disease in a “limited number” of patients, but has “no demonstrable survival benefits in patients with recurrent disease”.

Treatment for brain gliomas depends on the location, the cell type and the grade of malignancy. Histological diagnosis is mandatory, except in rare cases where biopsy or surgical resection is too dangerous. Often, treatment is a combined approach, using surgery, radiation therapy, and chemotherapy. The choice of treatments depends mainly on the histological study including the grading of the tumor. But unfortunately, the histological grading remains partly subjective and not always reproducible. Therefore, it is essential to define most relevant biological criteria to better adapt the treatments.

Blood vessels that make up the cardiovascular system may be broadly divided into arteries, veins and capillaries. Arteries carry blood away from the heart at relatively high pressure; veins carry blood back to the heart at low pressure, while capillaries provide the link between the arterial and venous blood supply. During embryonic development, vessels are first formed through vasculogenesis, utilizing pluripotent endothelial cell precursors. Later, through arteriogenesis, larger blood vessels are formed possessing a more complex structure of endothelial cells, smooth muscle cells and pericytes (tunica media). Although arteriogenesis is not considered to occur in the adult, blood vessels may be formed in the adult through vasculogenesis and notably a process known as angiogenesis. Under normal conditions, angiogenic neovascularization occurs during such conditions as wound repair, ischemic restoration and the female reproductive cycle (generating endometrium forming the corpus luteum and during pregnancy to create the placenta). The capillaries, relatively simple vessels formed by angiogenesis, lack a developed tunica as they are predominantly composed of endothelial cells and to a lesser extent perivascular cells and basement membrane. Cancer is a disease state characterized by the uncontrolled proliferation of altered tissue cells. Tumors less than a few millimeters in size utilize nearby normal vessels to provide nutrients and oxygen. However, above this critical size, cancer cells utilize angiogenesis to create additional vascular support. Normally, angiogenesis is kept in check by the body naturally creating angiogenic inhibitors to counteract angiogenic factors. However, the cancer cell changes this balance by producing angiogenic growth factors in excess of the angiogenic inhibitors, thus favoring blood vessel growth. Cancer initiated angiogenesis is not unlike angiogenesis observed during normal vessel growth. Angiogenic factors pass from the tumor cell to the normal endothelium, binding the endothelial cell, activating it and inducing endothelial signaling events leading to endothelial cell proliferation. Endothelial tubes begin to form, homing in toward the tumor with the formation of capillary loops. Capillaries then undergo a maturation process to stabilize loop structure. Cancer is but one disease associated with a pathological neovasculature. A wide variety of diseases involving aberrant angiogenesis exist in nature. These diseases utilize the same steps involved in normal capillary growth but do so in an aberrant manner creating capillaries which lack a high degree of stability and function. Agents capable of inhibiting angiogenesis would be expected to exert activity on a variety of pathological neovascular diseases. To date, antiangiogenic treatments have had limited success. It is the object of the current invention to utilize amniotic fluid stem cells as a differentiation stimuli to reduce neoplastic properties of glioblastoma and other brain tumors. An additional object of the invention is utilization of amniotic fluid stem cells to immune responses towards glioblastoma cells themselves, has well as associated endothelial cells and tumor stroma.

### SUMMARY

Various aspects of the invention are enumerated in the following paragraphs:

The invention embodies devices and methods of providing a small, quantity of a therapeutic gas mixture to be self-administered by patients in a non-clinical setting. The device is sufficiently safe and effective to meet the standards of the FDA (in the USA) and [other regulatory bodies in ex-US territories] for home use. According to preferred embodiments, the device is compact (e.g., can be entirely held and operated in one hand of the using patient), rugged, durable and portable, as well as simple and safe for the user to operate, especially in stressful environments, especially without the need for a medical provider being present to administer.

Preferred methods herein comprise: administering a compressed noble gas or nitrous oxide gas mixture to a patient in need comprising: identifying a patient in need of the gas mixture, such that they are suffering from a condition selected from the group consisting of: neurological disorders, anxiety depression, pain relief, inflammation, and stress disorders, providing a portable, handheld, gas delivery system, said gas delivery system comprising: a sealed pressurized cartridge containing a predetermined amount of compressed therapeutic gas mixture comprising either nitrous oxide or a noble gas mixture; a demand regulator comprising: (a) a cartridge receiver configured to releasably couple with the pressurized cartridge in a sealed connection with the regulator and allow intake of the gas mixture into the regulator based on the inhalation type of the patient; (b) an inhalation device; and (c) an outtake, where said demand regulator components are configured such that the inhalation of the patient through the inhalation device actuates the release of a portion of the gas mixture from the pressurized cartridge through the cartridge receiver into the regulator while lowering the pressure of the gas mixture within the regulator to ambient pressure, or substantially so, and then allows the gas mixture to enter into the patient's lungs from the regulator through the inhalation device; and further allows the patient's exhalation through the inhalation device to be released from the regulator through the outtake; and instructions from a medical provider to the patient directing a breath type during use; and the patient inhaling an amount the gas mixture through the inhalation device in accordance with the breath type provided in the instructions in an amount sufficient to alleviate said condition.

Preferred devices herein comprise a portable, handheld, gas delivery system, said gas delivery comprising: i) a sealed pressurized cartridge containing a predetermined amount of compressed therapeutic gas mixture comprising either nitrous oxide or a noble gas mixture; ii) a demand regulator comprising: (a) a cartridge receiver configured to releasably couple with the pressurized cartridge in a sealed connection with the regulator and allow intake of the gas mixture into the regulator based on the inhalation type of the patient; (b) an inhalation device; and (c) an outtake, where said demand regulator components are configured such that the inhalation of the patient through the inhalation device actuates the release of a portion of the gas mixture from the pressurized cartridge through the cartridge receiver into the regulator while lowering the pressure of the gas mixture within the regulator to ambient pressure, or substantially so, and then allows the gas mixture to enter into the patient's lungs from the regulator through the inhalation device; and further allows the patient's exhalation through the inhalation device to be released from the regulator through the outtake; and iii) instructions from a medical provider to the patient directing a breath type during use.

### SUMMARY

Embodiments herein relate to methods of treating glioma comprising the steps of: a) identifying a patient suffering from glioma; b) obtaining amniotic fluid; c) extracting from said amniotic fluid a population of cells with ability in inhibit neoplastic activity of glioma or other brain neoplasms; d) expanding said amniotic fluid cells with ability in inhibit neoplastic activity of glioma or other brain neoplasms in a manner to allow for increased number of cells while maintaining said ability inhibit neoplastic activity of glioma or other brain neoplasms, optionally treating said cells under conditions resembling the tumor microenvironment; and e) administering said expanded amniotic fluid derived cells into a patient suffering from glioma. Additional embodiments include the following:

Aspect 1. A method of treating glioma or other types of brain cancer by administering amniotic fluid stem cells which have been treated in a manner capable of stimulating immunogenicity.

Aspect 2. The method of Aspect 1, wherein said stimulation of immunogenicity of said amniotic fluid stem cells is achieved through culture with an agent capable of upregulating HLA expression.

Aspect 3. The method of Aspect 2, wherein said stimulation of immunogenicity of said amniotic fluid stem cells is achieved through culture with an agent capable of upregulating expression of costimulatory molecules.

Aspect 4. The method of Aspect 2, wherein said agent capable of stimulating upregulation of HLA expression induces activation of NF-kappa B.

Aspect 5. The method of Aspect 4, wherein said agent capable of inducing activation of NF-kappa B is an inhibitor of i-kappa B.

Aspect 6. The method of Aspect 2, wherein said agent capable of inducing upregulation of HLA is an activator of the JAK-STAT pathway.

Aspect 7. The method of Aspect 6, wherein said agent capable of activing the JAK-STAT pathway is interferon gamma.

Aspect 8. The method of Aspect 7, wherein said interferon gamma is added to culture of said amniotic fluid stem cells at a concentration of approximately 100 IU per ml for a period of approximately 48 hours.

Aspect 9. The method of Aspect 1, wherein said induction of immunogenicity in said amniotic fluid stem cells is achieved through treatment of said amniotic fluid stem cells with an agent capable of inducing signaling through a toll like receptor.

Aspect 10. The method of Aspect 1, wherein said induction of immunogenicity in said amniotic fluid stem cells is achieved through treatment of said amniotic fluid stem cells with an agent capable of inducing signaling through a Pathogen Associated Molecular Pattern (PAMP) receptor.

Aspect 11. The method of Aspect 10, wherein said PAMP receptor is selected from a group comprising of: a) MDA5; b) RIG-1; and c) NOD.

Aspect 12. The method of Aspect 9, wherein said toll like receptor is TLR-2.

Aspect 13. The method of Aspect 12, wherein said TLR-2 is activated by compounds selected from a group comprising of: a) Pam3cys4; b) Heat Killed Listeria monocytogenes (HKLM); and c) FSL-1.

Aspect 14. The method of Aspect 9, wherein said toll like receptor is TLR-3.

Aspect 15. The method of Aspect 14, wherein said TLR-3 is activated by Poly IC.

Aspect 16. The method of Aspect 14, wherein said TLR-3 is activated by double stranded RNA.

Aspect 17. The method of Aspect 16, wherein said double stranded RNA is of mammalian origin.

Aspect 18. The method of Aspect 16, wherein said double stranded RNA is of prokaryotic origin.

Aspect 19. The method of Aspect 16, wherein said double stranded RNA is derived from leukocyte extract.

Aspect 20. The method of Aspect 19, wherein said leukocyte extract is a heterogeneous composition derived from freeze-thawing of leukocytes, followed by dialysis for compounds less than 15 kDa.

Aspect 21. The method of Aspect 9, wherein said toll like receptor is TLR-4.

Aspect 22. The method of Aspect 21, wherein said TLR-4 is activated by lipopolysaccharide.

Aspect 23. The method of Aspect 21, wherein TLR-4 is activated by peptide possessing at least 80 percent homology to the sequence EFDVILKAAGANKVAVIKAVRGATGLGLKEAKDLVESAPAALKEGVSKDDAEALKKAL EEAGAEVEVK. (SEQ ID NO. 1)

Aspect 24. The method of Aspect 21, wherein said TLR-4 is activated by HMGB-1.

Aspect 25. The method of Aspect 24, wherein said HMGB-1 peptide is hp91.

Aspect 26. The method of Aspect 9, wherein said toll like receptor is TLR-5.

Aspect 27. The method of Aspect 26, wherein said TLR-5 is activated by flagellin.

Aspect 28. The method of Aspect 9, herein said toll like receptor is TLR-7.

Aspect 29. The method of Aspect 28, wherein said TLR-7 is activated by imiquimod.

Aspect 30. The method of Aspect 9, wherein said toll like receptor is TLR-8.

Aspect 31. The method of Aspect 30, wherein said TLR-8 is activated by resmiqiumod.

Aspect 32. The method of Aspect 9, wherein said toll like receptor is TLR-9

Aspect 33. The method of Aspect 32, wherein said TLR-9 is activated by CpG DNA.

Aspect 34. The method of Aspect 1, wherein conventional cancer therapeutics are added to vaccination with amniotic fluid stem cells whose immunogenicity has been augmented.

Aspect 35. The method of Aspect 34, wherein conventional cancer therapies comprise of PACE (prednisone, doxorubicin, cyclophosphamide, and etoposide), CHOP (cyclophosphamide, doxorubicin, vincristine, and prednisone), CHOP-R (cyclophosphamide, doxorubicin, vincristine, prednisone, rituximab), B-R (bendamustine and rituximab), CVP (cyclophosphamide, vincristine, and prednisone), CVP-R (cyclophosphamide, vincristine, prednisone, and rituximab), F-R (fluradarabine and rituximab), FND-R (fludarabine, mitoxantrone, dexamethasone, and rituximab), FCM (fludarabine, cyclophosphamide, and mitoxantrone), FCM-R (fludarabine, cyclophosphamide, mitoxantrone, and rituximab), radioimmunotherapy, single agent rituximab, single agent alkylator, lenalidomide, involved field radiation therapy, or stem cell transplant.

### BRIEF DESCRIPTION OF THE DRAWINGS

FIG. 1 is a bar graph showing proliferation of various types and concentrations of exosomes.

### DETAILED DESCRIPTION OF THE INVENTION

The invention teaches the previously unknown property of amniotic fluid stem cells and exosomes derived thereof as a means of reducing neoplastic properties of glioma and other brain cancers. In one particular embodiment, patients suffering from glioma are administered amniotic fluid stem cells initravenously, or locally in proximity to the tumors. In one particular embodiment amniotic fluid stem cells are administered intrathecally. In another embodiment amniotic fluid stem cells are administered intraventricularly.

In one embodiment of the invention cells derived from amniotic fluid are used for treatment of cytokine storm. The cells described in the invention are immortal in culture, maintain euploidy for >1 year in culture, share markers with human ES cells, and are capable of differentiating into all three germ layers of the developing embryo, Endoderm, Mesoderm and Ectoderm. In a preferred embodiment the regenerative amniotic fluid cells are found in the amnion harvested during the second trimester of human pregnancies. It is known that amniotic fluid contains multiple morphologically-distinguishable cell types, the majority of the cells are prone to senescence and are lost from cultures. In one embodiment, fibronectin coated plates and culture conditions described in U.S. Pat. No. 7,569,385 are used to grow cells from amniotic fluid harvests from normal 16-18 week pregnancies. The cells of the invention are of fetal origin, and have a normal diploid karyotype. Growth of the amniotic fluid stem cells as described in the invention for use in neurological ischemic conditions results in cells that are multipotent, as several main cell types have been derived from them. As used herein, the term “multipotent” refers to the ability of amniotic fluid regenerative cells to differentiate into several main cell types. The MAFSC cells may also be propagated under specific conditions to become “pluripotent.” The term “pluripotent stem cells” describes stem cells that are capable of differentiating into any type of body cell, when cultured under conditions that give rise to the particular cell type. The Amniotic fluid regenerative cells are preferably isolated from humans. However, the Amniotic fluid regenerative cells may be isolated in a similar manner from other species. Examples of species that may be used to derive the Amniotic fluid regenerative cells include but are not limited to mammals, humans, primates, dogs, cats, goats, elephants, endangered species, cattle, horses, pigs, mice, rabbits, and the like.

The amniotic fluid-derived cells and MAFSC can be recognized by their specific cell surface proteins or by the presence of specific cellular proteins. Typically, specific cell types have specific cell surface proteins. These surface proteins can be used as “markers” to determine or confirm specific cell types. Typically, these surface markers can be visualized using antibody-based technology or other detection methods. One method of characterizing cellular markers, FACS analysis, is described in Example 3.

The surface markers of the isolated MAFSC cells derived from independently-harvested amniotic fluid samples were tested for a range of cell surface and other markers, using monoclonal antibodies and FACS analysis. These cells can be characterized by the following cell surface markers: SSEA3, SSEA4, Tra-1-60, Tra-1-81, Tra-2-54, as shown in FIG. 3. The MAFSC cells can be distinguished from mouse ES cells in that the MAFSC cells do not express the cell surface marker SSEA1. Additionally, MAFSC express the stem cell transcription factor Oct-4. The MAFSC cells can be recognized by the presence of at least one, or at least two, or at least three, or at least four, or at least five, or at least six, or all of the following cellular markers SSEA3, SSEA4, Tra-1-60, Tra-1-81, Tra-2-54 and Oct-4.

In some embodiments of the present invention, the SSEA3 marker is expressed in a range of from about 90%, 92%, 94% to about 96%, 98%, 99%, or 100% of the cells in the MAFSC culture. The SSEA4 marker can be expressed, for example, in a range of from about 90%, 92%, 94% to about 96%, 98%, 99%, or 100% of the cells in the MAFSC culture. In some embodiments of the present invention, the Tra-1-60 marker expressed, for example, in a range of from about 60%, 65%, or 70% to about 85%, 90%, or 95% of the cells in the MAFSC culture. In some embodiments of the present invention, the Tra-1-81 marker is expressed in a range of from about 70%, 75%, or 80% to about 85%, 90%, or 95% of the cells in the MAFSC culture. The Tra-2-84 marker can be expressed, for example, in a range of from about 55%, 60%, 65%, or 70% to about 80%, 90%, or 95% of the cells in the MAFSC culture. In some embodiments of the present invention, the Oct-4 marker is expressed in a range of from about 25%, 30%, 35%, or 40% to about 45%, 55%, 65%, or 70% of the cells in the MAFSC culture.

The MAFSC cultures express very little or no SSEA-1 marker. In addition to the embryo stem cell markers SSEA3, SSEA4, Tra1-60, Tra1-81, Tra2-54, Oct-4 the amniotic fluid regenerative cells also expressed high levels of the cell surface antigens that are normally found on human mesenchymal stem cells, but not normally on human embryo stem cells (M F Pittinger et al., Science 284:143-147, 1999; S Gronthos et al., J. Cell Physiol. 189:54-63, 2001). This set of markers includes CD13 (99.6%) aminopeptidase N, CD44 (99.7%) hyaluronic acid-binding receptor, CD49b (99.8%) collagen/laminin-binding integrin alpha2, and CD105 (97%) endoglin. The presence of both the embryonic stem cell markers and the hMSC markers on the MAFSC cell cultures indicates that amniotic fluid-derived MAFSC cells, grown and propagated as described here, represent a novel class of human stem cells that combined the characteristics of hES cells and of hMSC cells.

In some embodiments of the invention, at least about 90%, 94%, 97%, 99%, or 100% of the cells in the culture express CD13. In additional embodiments, at least about 90%, 94%, 97%, 99%, or 100% of the cells in the culture express CD44. In some embodiments of the invention, a range from at least about 90%, 94%, 97%, 99%, 99.5%, or 100% of the cells in the culture express CD49b. In further embodiments of the invention, a range from at least about 90%, 94%, 97%, 99%, 99.5%, or 100% of the cells in the culture express CD105.

In a particularly advantageous embodiment, the amniotic fluid regenerative cells are human stem cells that can be propagated for an indefinite period of time in continuous culture in an undifferentiated state. The term “undifferentiated” refers to cells that have not become specialized cell types. A “nutrient medium” is a medium for culturing cells containing nutrients that promote proliferation. The nutrient medium may contain any of the following in an appropriate combination: isotonic saline, buffer, amino acids, antibiotics, serum or serum replacement, and exogenously added factors.

Administration of MAFSC, or amniotic fluid stem cells (the terms are used herein interchangeably) is performed, in one embodiment, intravenously, at concentrations sufficient to reduce neoplastic ability of glioma or other brain cancer cells.

Any medium capable of supporting MSC in vitro may be used to culture the amniotic fluid stem cells. Media formulations that can support the growth of MSC include, but are not limited to, Dulbecco's Modified Eagle's Medium (DMEM), alpha modified Minimal Essential Medium (.alpha.MEM), and Roswell Park Memorial Institute Media 1640 (RPMI Media 1640) and the like. Said media and conditions for culture of MSC—and by virtue of the invention MSC are known in the art. Typically, up to 20% fetal bovine serum (FBS) or 1-20% horse serum is added to the above medium in order to support the growth of MSC. A defined medium, however, also can be used if the growth factors, cytokines, and hormones necessary for culturing MSC are provided at appropriate concentrations in the medium. Media useful in the methods of the invention may contain one or more compounds of interest, including, but not limited to, antibiotics, mitogenic or differentiation compounds useful for the culturing of MSC. The cells may be grown at temperatures between 27.degree. C. to 40.degree. C., preferably 31.degree. C. to 37.degree. C., and more preferably in a humidified incubator. The carbon dioxide content may be maintained between 2% to 10% and the oxygen content may be maintained between 1% and 22%. The invention, however, should in no way be construed to be limited to any one method of isolating and culturing MSC. Rather, any method of isolating and culturing MSC should be construed to be included in the present invention. Antibiotics which can be added into the medium include, but are not limited to, penicillin and streptomycin. The concentration of penicillin in the culture medium, in a non-limiting embodiment, is about 10 to about 200 units per ml. The concentration of streptomycin in the culture medium is, in a non-limiting embodiment, about 10 to about 200 .mu.g/ml.

Amniotic fluid stem cells may be administered to an animal in an amount effective to provide a therapeutic effect. The animal may be a mammal, including but not limited to, human and non-human primates. The MSC can be suspended in an appropriate diluent. Suitable excipients for injection solutions are those that are biologically and physiologically compatible with the MSC and with the recipient, such as buffered saline solution or other suitable excipients. The composition for administration can be formulated, produced, and stored according to standard methods complying with proper sterility and stability. The MSC may have one or more genes modified or be treated such that the modification has the ability to cause the MSC to self-destruct or “commit suicide” because of such modification, or upon presentation of a second drug (eg., a prodrug) or signaling compound to initiate such destruction of the MSC.

Furthermore, the invention provides a therapeutic composition useful for stimulation of immunity against proliferating endothelium, with particular emphasis on glioma and brain neoplasms. In one specific embodiment of the invention, amniotic fluid stem cells are treated with agents capable of augmenting immunogenicity, and subsequently administered into a recipient in which immune response to proliferating endothelium is desired. In one specific example, amniotic fluid stem cells are purified and expanded under conditions capable of augmenting immunogenicity. Said immunogenicity in this context refers to ability to enhance recognition by recipient immune system. In one embodiment, immunogenicity refers to enhanced expression of HLA I and/or HLA II molecules. In another embodiment, immunogenicity refers to enhanced expression of costimulatory molecules. Said costimulatory molecules are selected from a group comprising of: CD27; CD80; CD86; ICOS; OX-4; and 4-1 BB. In another embodiment, immunogenicity refers to enhanced ability to stimulate proliferation of allogeneic lymphocytes in a mixed lymphocyte reaction. Immunogenicity may be augmented by incubation with one of the lymphokine or cytokine proteins that are known in the art, or with a member of the interferon family. In one particular embodiment, said purified amniotic fluid cells are incubated with interferon gamma. In one particular embodiment, interferon gamma is incubated with amniotic fluid cells, whether purified or unpurified for a period of approximately 48 hours, at a concentration of approximately 150 IU/ml. Amniotic fluid cells may be expanded after purification as described above before treatment with agents capable of augmenting immunogenicity. For example, cells may be treated with a cell mitogen. Said cell mitogen may be any protein, polypeptide, variant or portion thereof that is capable of, directly or indirectly, inducing endothelial cell growth. Such proteins include, for example, acidic and basic fibroblast growth factors (aFGF) (GenBank Accession No. NP.sub.--149127) and bFGF (GenBank Accession No. AAA52448), vascular endothelial growth factor (VEGF) (GenBank Accession No. AAA35789 or NP.sub.--001020539), epidermal growth factor (EGF) (GenBank Accession No. NP.sub.--001954), transforming growth factor .alpha. (TGF-.alpha.) (GenBank Accession No. NP.sub.--003227) and transforming growth factor .beta. (TFG-.beta.) (GenBank Accession No. 1109243A), platelet-derived endothelial cell growth factor (PD-ECGF) (GenBank Accession No. NP.sub.--001944), platelet-derived growth factor (PDGF) (GenBank Accession No. 1109245A), tumor necrosis factor .alpha. (TNF-.alpha.) (GenBank Accession No. CAA26669), hepatocyte growth factor (HGF) (GenBank Accession No. BAA14348), insulin like growth factor (IGF) (GenBank Accession No. P08833), erythropoietin (GenBank Accession No. P01588), colony stimulating factor (CSF), macrophage-CSF (M-CSF) (GenBank Accession No. AAB59527), granulocyte/macrophage CSF (GM-CSF) (GenBank Accession No. NP.sub.--000749), monocyte chemotactic protein-1 (GenBank Accession No. P13500) and nitric oxide synthase (NOS) (GenBank Accession No. AAA36365). See, Klagsbrun, et al., Annu. Rev. Physiol., 53:217-239 (1991); Folkman, et al., J. Biol. Chem., 267:10931-10934 (1992) and Symes, et al., Current Opinion in Lipidology, 5:305-312 (1994). Variants or fragments of a mitogen may be used as long as they induce or promote amniotic fluid stem cell growth. Preferably, the cell mitogen contains a secretory signal sequence that facilitates secretion of the protein. Proteins having native signal sequences, e.g., VEGF, are preferred. Proteins that do not have native signal sequences, e.g., bFGF, can be modified to contain such sequences using routine genetic manipulation techniques. See, Nabel et al., Nature, 362:844 (1993). Before expansion, cells may be further purified based on expression of surface receptors using affinity-based methodologies that are known to one of skill in the art, said methodologies include magnetic activated cell sorting (MACS), cell panning, or affinity chromatography. Other methodologies such as fluorescent activated cell sorting (FACS) may also be used. Various lectins are known to have selectivity to cells, for example, Ulex europaeus agglutinin I is known to possess ability to bind to amniotic fluid stem cells.

The cancer vaccine formulation may be utilized in conjunction with known adjuvants in order to induce an immune response that is Th1 or Th17-like, and which will inhibit the proliferation of endothelial cells in the recipient. Such adjuvant compounds are known in the art to boost the activity of the immune system and are now under study as possible adjuvants, particularly for vaccine therapies. Some of the most commonly studied adjuvants are listed below, but many more are under development. For example, Levamisole, a drug originally used against parasitic infections, has recently been found to improve survival rates among people with colorectal cancer when used together with some chemotherapy drugs [2-8]. It is often used as an immunotherapy adjuvant because it can activate T lymphocytes [9-11]. Additionally, the compound has been demonstrated to induce maturation of dendritic cells, further supporting an immune modulatory role [12]. Levamisole is now used routinely for people with some stages of colorectal cancer and is being tested in clinical trials as a treatment for other types of cancer. Additionally, it has been shown to augment efficacy of other immunotherapeutic agents such as interferon [13, 14]. Aluminum hydroxide (alum) is one of the most common adjuvants used in clinical trials for cancer vaccines. It is already used in vaccines against several infectious agents, including the hepatitis B virus. Bacille Calmette-Guerin (BCG) is a bacterium that is related to the bacterium that causes tuberculosis. The effect of BCG infection on the immune system makes this bacterium useful as a form of anticancer immunotherapy [15]. BCG was one of the earliest immunotherapies used against cancer, either alone, or in combination with other therapies such as hormonal, chemotherapy or radiotherapy [16-24]. It is FDA approved as a routine treatment for superficial bladder cancer. Its usefulness in other cancers as a nonspecific adjuvant is also being tested or has demonstrated therapeutic effects [25-33]. Researchers are looking at injecting BCG to give an added stimuli to the immune system when using chemotherapy, radiation therapy, or other types of immunotherapy. Thus in various embodiments of the current invention, one of skill in the art is directed towards references which have utilized BCG as an adjuvant for other therapies for concentrations and dosing regimens that would apply to the current invention for elicitation of immunity towards proliferating endothelial cells. Incomplete Freund's Adjuvant (IFA) is given together with some experimental therapies to help stimulate the immune system and to increase the immune response to cancer vaccines, both protein and peptide in part by providing a localization factor for T cells [34-42]. IFA is a liquid consisting of an emulsifier in white mineral oil. Another vaccine adjuvant useful for the present invention is interferon alpha, which has been demonstrated to augment NK cell activity, as well as to promote T cell activation and survival [43]. QS-21 is a relatively new immune stimulant made from a plant extract that increases the immune response to vaccines used against melanoma. DETOX is another relatively new adjuvant. It is made from parts of the cell walls of bacteria and a kind of fat. It is used with various immunotherapies to stimulate the immune system. Keyhole limpet hemocyanin (KLH) is another adjuvant used to boost the effectiveness of cancer vaccine therapies. It is extracted from a type of sea mollusc. Dinitrophenyl (DNP) is a hapten/small molecule that can attach to tumor antigens and cause an enhanced immune response. It is used to modify tumor cells in certain cancer vaccines.

In one embodiment of the invention proliferating amniotic fluid stem cells are treated with an agent to stimulate immunogenicity are lysed and protein extracts are extracted and utilized as a vaccine. In some embodiments, specific immunogenic peptides may be isolated for said cell lysate. In other embodiments, lyophilization of amniotic fluid stem cells is performed subsequent to treatment with an agent that augments immunogenicity. In embodiments utilizing cellular extracts, various formulations may be generated. The formulations may conveniently be presented in unit dosage form and may be prepared by any of the methods well known in the art of pharmacy. Such methods include the step of bringing into association the active ingredient (for an antigenic molecule, construct or chimeric polypeptide of the invention) with the carrier which constitutes one or more accessory ingredients. In general, the formulations are prepared by uniformly and intimately bringing into association the active ingredient with liquid carriers or finely divided solid carriers or both, and then, if necessary, shaping the product.

Formulations in accordance with the present invention suitable for oral administration may be presented as discrete units such as capsules, cachets or tablets, each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste. In situations where an orally available vaccine is desirable, a tablet may be made by compression or moulding, optionally with one or more accessory ingredients. Compressed tablets may be prepared by compressing in a suitable machine the active ingredient in a free-flowing form such as a powder or granules, optionally mixed with a binder (eg povidone, gelatin, hydroxypropylmethyl cellulose), lubricant, inert diluent, preservative, disintegrant (eg sodium starch glycolate, cross-linked povidone, cross-linked sodium carboxymethyl cellulose), surface-active or dispersing agent. Moulded tablets may be made by moulding in a suitable machine a mixture of the powdered compound moistened with an inert liquid diluent. The tablets may optionally be coated or scored and may be formulated so as to provide slow or controlled release of the active ingredient therein using, for example, hydroxypropylmethylcellulose in varying proportions to provide desired release profile. Formulations suitable for topical administration in the mouth include lozenges comprising the active ingredient in a flavoured base, usually sucrose and acacia or tragacanth; pastilles comprising the active ingredient in an inert base such as gelatin and glycerin, or sucrose and acacia; and mouth-washes comprising the active ingredient in a suitable liquid carrier. Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules and tablets of the kind previously described. Nasal sprays may be useful formulations. Preferred unit dosage formulations are those containing a single or daily dose or unit, daily sub-dose or an appropriate fraction thereof, of an active ingredient.

It will be appreciated that the therapeutic molecule can be delivered to the locus by any means appropriate for localized administration of a drug. For example, a solution of the therapeutic molecule can be injected directly to the site or can be delivered by infusion using an infusion pump. The construct, for example, also can be incorporated into an implantable device which when placed at the desired site, permits the construct to be released into the surrounding locus. The therapeutic molecule may be administered via a hydrogel material. The hydrogel is non-inflammatory and biodegradable. Many such materials now are known, including those made from natural and synthetic polymers. In a preferred embodiment, the method exploits a hydrogel which is liquid below body temperature but gels to form a shape-retaining semisolid hydrogel at or near body temperature. Preferred hydrogel are polymers of ethylene oxide-propylene oxide repeating units. The properties of the polymer are dependent on the molecular weight of the polymer and the relative percentage of polyethylene oxide and polypropylene oxide in the polymer. Preferred hydrogels contain from about 10% to about 80% by weight ethylene oxide and from about 20% to about 90% by weight propylene oxide. A particularly preferred hydrogel contains about 70% polyethylene oxide and 30% polypropylene oxide. Hydrogels which can be used are available, for example, from BASF Corp., Parsippany, N.J., under the tradename Pluronic.sup.R. Although subcutaneous, intradermal, and intramuscular routes of administration are preferred, administration into lymphatics of the vaccine preparation is also envisioned within the scope of the current invention. Endpoints guiding the practitioner of the invention include: a) ability of the vaccine to stimulate immunity towards proliferating endothelial cells; b) ability of the vaccine to stimulate immunity towards cancer-associated molecules; and c) ability of the vaccine to stimulate immunity towards tumor cells.

Unless defined differently, all technical and scientific terms used herein have the same meanings as commonly understood by one of skill in the art to which the disclosed invention belongs. In particular, the following terms and phrases have the following meaning.

“Angiogenesis” means any alteration of an existing vascular bed or the formation of new vasculature which benefits tissue perfusion. This includes the formation of new vessels by sprouting of endothelial cells from existing blood vessels or the remodeling of existing vessels to alter size, maturity, direction or flow properties to improve blood perfusion of tissues. As used herein the terms, “angiogenesis,” “revascularization,” “increased collateral circulation,” and “regeneration of blood vessels” are considered as synonymous.

As used herein, “cancer” refers to all types of cancer or neoplasm or malignant tumors found in animals, including leukemias, carcinomas and sarcomas. Examples of cancers are cancer of the brain, melanoma, bladder, breast, cervix, colon, head and neck, kidney, lung, non-small cell lung, mesothelioma, ovary, prostate, sarcoma, stomach, uterus and Medulloblastoma. The term “leukemia” is meant broadly progressive, malignant diseases of the hematopoietic organs/systems and is generally characterized by a distorted proliferation and development of leukocytes and their precursors in the blood and bone marrow. Leukemia diseases include, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophilic leukemia, blast cell leukemia, bovine leukemia, chronic myelocytic leukemia, leukemia cutis, embryonal leukemia, eosinophilic leukemia, Gross' leukemia, Rieder cell leukemia, Schilling's leukemia, stem cell leukemia, subleukemic leukemia, undifferentiated cell leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, plasmacytic leukemia, and promyelocytic leukemi.

In some aspects of the invention, it will be important to overcome tolerance that already exists to proliferating self endothelial cells. Accordingly, on of skill in the art is directed towards the following description of tolerogenic processes, with the knowledge that manipulation and specific inhibition of these processes is useful in the practice of the current invention. The argument has been made that tolerance is controlled to some extent by immature dendritic cells presenting self antigen in absence of costimulation/presence of co-inhibitors, which leads to generation of Treg cells and anergic T cells [44]. This was demonstrated in several systems, for example, in a classical experiment Mahnke et al targeted the antigen ovalbumin to immature dendritic cells by conjugation to anti-DEC205 antibodies. It was demonstrated that antigen-specific Treg were generated, which was dependent on presentation by immature dendritic cells [45]. In vivo relevance of Treg generated by targeting antigen to steady state dendritic cells can be seen in studies where DEC-205 targeting of antigen prevented autoimmune diabetes in a transgenic model system via FoxP3 expressing Treg [46]. We have reported on a “tolerogenic vaccine” created by ex vivo generation of immature DC treated with a chemical IKK inhibitor, and pulsed with collagen II, that was able to prevent arthritis in a mouse model [47]. Similar tolerogenic uses of immature DC have been reported in diverse conditions such as transplantation [48], anti-Factor VIII immunity [49], autoimmune myocarditis, experimental autoimmune mysthenia gravis [50], and collagen induced arthritis [51]. The possibility that tumors may be generating immature DC to protect themselves from T cell attack and/or generate Treg was suggested in studies showing tumor secreted VEGF would arrest DC maturation in vitro [52]. Mechanistically it was demonstrated that VEGF blocks NF-kB activity in DC, which is a critical maturation-inducing factor [53]. Given that VEGF is a primary cytokine in tumor angiogenesis, the possibility of inhibited DC maturation being a mechanism of immune escape is attractive. Angiogenesis seems to be associated with various cells of the myeloid lineage. The myeloid suppressor cell, which will be described below, has been demonstrated stimulate angiogenesis directly, and through production of MMP-9 and VEGF [54]. In HNSCC a population of myeloid suppressor cells was described in a series of publications by Rita Young's group. These cells, which express the hematopoietic stem cell marker CD34, were originally identified as the source of intra-tumor GM-CSF detected from primary patient samples [55]. Suggesting a possible immune inhibitory role for these cells were data that their depletion results in upregulated ability of lymphocytes within the tumor to generate IL-2, which was lost upon re-introduction of these cells into culture. Clinical relevance of these myeloid suppressor cells was supported by a study of 20 HNSCC patients whose tumors were resected and relapsed, compared to 17 patients that had disease free survival for the 2-year observation period. Tumors of patients relapsed produced almost 4-fold higher levels of GM-CSF and had approximately 2.5-fold the number of CD34+ cells as compared to patients that were free of disease [56]. Mechanistic study of these cells revealed suppression of T cell activity could be abolished treatment with antibodies to TGF-b, and that inhibitory activity was lost upon their differentiation with agents such as IFN-g and TNF-alpha [57]. Given that immature DC mediate Treg generation through TGF-b [44], and that immature DC lose inhibitory activity upon maturation with agents such as IFN-g and TNF-alpha, the possible relationship with myeloid suppressor cells was considered [58]. In fact, a recent study suggested the possibility of vivo differentiation of myeloid suppressor cells. Newly diagnosed HNSCC patients were treated with Vitamin D3 for three weeks before surgical excision of the tumor. Observations of significant reduction in numbers of intratumoral CD34 cells and augmented numbers of dendritic cells were reported [59]. Other interventions for induction of myeloid suppressor cell differentiation into DC/reversing immune suppressive potential have demonstrated some promise including 5-azacytidine [60], sunitinib [61], PDE-5 inhibitors [62], and inhibitors of stem cell factor or its receptor c-kit [63]. Of these, 5-azaycytidine, sunitinib various PDE-5 inhibitors are already part of clinical practice. In the case of sunitinib, clinical evidence of derepression of T cell responses after therapy has been reported [64], effects being mediated, in part, by suppression of STAT3 activity [65]. Myeloid suppressor cells have been described in numerous other conditions of neoplasia, in which GM-CSF has been reported to be a major factor in their generation [66, 67]. In addition to TGF-beta, suppression by myeloid suppressor cells seems to be mediated by PGE-2 [68], expression of arginase, which generates immune suppressive polyamines [54], and depletion of cystine and cysteine [54] (amino acids needed for T cell activation). Thus while it is still not completely clear how upstream in the differentiation pathway myeloid suppressor cells are as compared to immature dendritic cells, there is evidence that both cell populations mediate generation of Treg cells [69]. In the case of HNSCC at least one paper supports in situ generation of Treg by immature antigen presenting cells, specifically, a study comparing SCC with Actinic Keratosis demonstrated that increased Treg cell numbers were associated with local DC, in SCC [70]. Others have made correlations between myeloid suppressor cell numbers and Treg [71-73]. Thus it is within the scope of the current invention to induce in vivo maturation/activation of DC, in order to augment breaking of self tolerance towards tumor tissue, with particular emphasis on tumor-associated endothelial cells. The injection for parenteral administration of the tumor-resembling endothelial cell immunogen, otherwise termed ValloVax may be an aqueous injection or an oily injection. The aqueous injection can be prepared according to a known method, for example, by appropriately adding a pharmaceutically acceptable additive to an aqueous solvent (water for injection, purified water, etc.) to make a solution, mixing the WT1 protein or WT1 peptide with the solution, filter sterilizing the resulting mixture with a filter etc., and then filling an aseptic container with the resulting filtrate. Examples of the pharmaceutically acceptable additive include the above-mentioned adjuvants; isotonizing agents such as sodium chloride, potassium chloride, glycerol, mannitol, sorbitol, boric acid, borax, glucose and propylene glycol; buffering agents such as a phosphate buffer solution, an acetate buffer solution, a borate buffer solution, a carbonate buffer solution, a citrate buffer solution, a Tris buffer solution, a glutamate buffer solution and an epsilon-aminocaproate solution; preservatives such as methyl parahydroxybenzoate, ethyl parahydroxybenzoate, propyl parahydroxybenzoate, butyl parahydroxybenzoate, chlorobutanol, benzyl alcohol, benzalkonium chloride, sodium dehydroacetate, sodium edetate, boric acid and borax; thickeners such as hydroxyethylcellulose, hydroxypropylcellulose, polyvinyl alcohol and polyethylene glycol; stabilizers such as sodium hydrogen sulfite, sodium thiosulfate, sodium edetate, sodium citrate, ascorbic acid and dibutyl hydroxy toluene; and pH adjusters such as hydrochloric acid, sodium hydroxide, phosphoric acid and acetic acid. The injection may further contain an appropriate solubilizing agent, and examples thereof include alcohols such as ethanol; polyalcohols such as propylene glycol and polyethylene glycol; and non-ionic surfactants such as polysorbate 80, polyoxyethylene hydrogenated castor oil 50, lysolecithin and pluronic polyols. Also, proteins such as bovine serum albumin and keyhole limpet hemocyanin; polysaccharides such as aminodextran; etc. may be contained in the injection. For preparation of the oily injection, for example, sesame oil or soybean oil is used as an oily solvent, and benzyl benzoate or benzyl alcohol may be blended as a solubilizing agent. The prepared injection is usually stored in an appropriate ampule, vial, etc. The liquid preparations, such as injections, can also be deprived of moisture and preserved by cryopreservation or lyophilization. The lyophilized preparations become ready to use by redissolving them in added distilled water for injection etc. just before use.

In some embodiments of the invention exosomes or microvesicles derived from amniotic fluid stem cells are administered instead of amniotic fluid stem cells for generation of glioma inhibitory properties.

In one embodiment, fibroblasts are cultured using means known in the art for preserving viability and proliferative ability of fibroblasts. The invention may be applied both for individualised autologous exosome preparations and for exosome preparations obtained from established cell lines, for experimental or biological use. In one embodiment, this invention is more specifically based on the use of chromatography separation methods for preparing membrane vesicles, particularly to separate the membrane vesicles from potential biological contaminants, wherein said microvesicles are exosomes, and cells utilized for generating said exosomes are fibroblast cells.

Indeed, the applicant has now demonstrated that membrane vesicles, particularly exosomes, could be purified, and possess ability to stimulate angiogenesis. In one embodiment, a strong or weak, preferably strong, anion exchange may be performed. In addition, in a specific embodiment, the chromatography is performed under pressure. Thus, more specifically, it may consist of high performance liquid chromatography (HPLC). Different types of supports may be used to perform the anion exchange chromatography. More preferably, these may include cellulose, poly(styrene-divinylbenzene), agarose, dextran, acrylamide, silica, ethylene glycol-methacrylate co-polymer, or mixtures thereof, e.g., agarose-dextran mixtures. To illustrate this, it is possible to mention the different chromatography equipment composed of supports as mentioned above, particularly the following gels: SOURCE®, POROS®, SEPHAROSE®, SEPHADEX®, TRISACRYL®, TSK-GEL SW™, OR PW®, SUPERDEX®, TOYOPEARL HW™ and SEPHACRYL®, for example, which are suitable for the application of this invention. Therefore, in a specific embodiment, this invention relates to a method of preparing membrane vesicles, particularly exosomes, from a biological sample such as a tissue culture containing fibroblasts, comprising at least one step during which the biological sample is treated by anion exchange chromatography on a support selected from cellulose, poly(styrene-divinylbenzene), silica, acrylamide, agarose, dextran, ethylene glycol-methacrylate co-polymer, alone or in mixtures, optionally functionalised.

In addition, to improve the chromatographic resolution, within the scope of the invention, it is preferable to use supports in bead form. Ideally, these beads have a homogeneous and calibrated diameter, with a sufficiently high porosity to enable the penetration of the objects under chromatography (i.e. the exosomes). In this way, given the diameter of exosomes (generally between 50 and 100 nm), to apply the invention, it is preferable to use high porosity gels, particularly between 10 nm and 5 .mu.m, more preferably between approximately 20 nm and approximately 2 .mu.m, even more preferably between about 100 nm and about 1 .mu.m. For the anion exchange chromatography, the support used must be functionalised using a group capable of interacting with an anionic molecule. Generally, this group is composed of an amine which may be ternary or quaternary, which defines a weak or strong anion exchanger, respectively. Within the scope of this invention, it is particularly advantageous to use a strong anion exchanger. In this way, according to the invention, a chromatography support as described above, functionalised with quaternary amines, is used. Therefore, according to a more specific embodiment of the invention, the anion exchange chromatography is performed on a support functionalised with a quaternary amine. Even more preferably, this support should be selected from poly(styrene-divinylbenzene), acrylamide, agarose, dextran and silica, alone or in mixtures, and functionalised with a quaternary amine. Examples of supports functionalised with a quaternary amine include the gels SOURCEQ. MONO Q, Q SEPHAROSE®, POROS®, HQ and POROS, QE, FRACTOGEL®, TMAE type gels and TOYOPEARL™ SUPER® Q™ gels.

A particularly preferred support to perform the anion exchange chromatography comprises poly(styrene-divinylbenzene). An example of this type of gel which may be used within the scope of this invention is SOURCE Q gel, particularly SOURCE 15 Q (Pharmacia). This support offers the advantage of very large internal pores, thus offering low resistance to the circulation of liquid through the gel, while enabling rapid diffusion of the exosomes to the functional groups, which are particularly important parameters for exosomes given their size. The biological compounds retained on the column may be eluted in different ways, particularly using the passage of a saline solution gradient of increasing concentration, e.g. from 0 to 2 M. A sodium chloride solution may particularly be used, in concentrations varying from 0 to 2 M, for example. The different fractions purified in this way are detected by measuring their optical density (OD) at the column outlet using a continuous spectro-photometric reading. As an indication, under the conditions used in the examples, the fractions comprising the membrane vesicles were eluted at an ionic strength comprised between approximately 350 and 700 mM, depending on the type of vesicles.

Different types of columns may be used to perform this chromatographic step, according to requirements and the volumes to be treated. For example, depending on the preparations, it is possible to use a column from approximately 100 .mu.l up to 10 ml or greater. In this way, the supports available have a capacity which may reach 25 mg of proteins/ml, for example. For this reason, a 100 .mu.l column has a capacity of approximately 2.5 mg of proteins which, given the samples in question, allows the treatment of culture supernatants of approximately 2 l (which, after concentration by a factor of 10 to 20, for example, represent volumes of 100 to 200 ml per preparation). It is understood that higher volumes may also be treated, by increasing the volume of the column, for example. In addition, to perform this invention, it is also possible to combine the anion exchange chromatography step with a gel permeation chromatography step. In this way, according to a specific embodiment of the invention, a gel permeation chromatography step is added to the anion exchange step, either before or after the anion exchange chromatography step. Preferably, in this embodiment, the permeation chromatography step takes place after the anion exchange step. In addition, in a specific variant, the anion exchange chromatography step is replaced by the gel permeation chromatography step. The present application demonstrates that membrane vesicles may also be purified using gel permeation liquid chromatography, particularly when this step is combined with an anion exchange chromatography or other treatment steps of the biological sample, as described in detail below.

To perform the gel permeation chromatography step, a support selected from silica, acrylamide, agarose, dextran, ethylene glycol-methacrylate co-polymer or mixtures thereof, e.g., agarose-dextran mixtures, are preferably used. As an illustration, for gel permeation chromatography, a support such as SUPERDEX® 200HR (Pharmacia), TSK G6000 (TosoHaas) or SEPHACRYL® S (Pharmacia) is preferably used. The process according to the invention may be applied to different biological samples. In particular, these may consist of a biological fluid from a subject (bone marrow, peripheral blood, etc.), a culture supernatant, a cell lysate, a pre-purified solution or any other composition comprising membrane vesicles.

In this respect, in a specific embodiment of the invention, the biological sample is a culture supernatant of membrane vesicle-producing fibroblast cells.

In addition, according to a preferred embodiment of the invention, the biological sample is treated, prior to the chromatography step, to be enriched with membrane vesicles (enrichment stage). In this way, in a specific embodiment, this invention relates to a method of preparing membrane vesicles from a biological sample, characterised in that it comprises at least: b) an enrichment step, to prepare a sample enriched with membrane vesicles, and c) a step during which the sample is treated by anion exchange chromatography and/or gel permeation chromatography.

In one embodiment, the biological sample is a culture supernatant treated so as to be enriched with membrane vesicles. In particular, the biological sample may be composed of a pre-purified solution obtained from a culture supernatant of a population of membrane vesicle-producing cells or from a biological fluid, by treatments such as centrifugation, clarification, ultrafiltration, nanofiltration and/or affinity chromatography, particularly with clarification and/or ultrafiltration and/or affinity chromatography. Therefore, a preferred method of preparing membrane vesicles according to this invention more particularly comprises the following steps: a) culturing a population of membrane vesicle (e.g. exosome) producing cells under conditions enabling the release of vesicles, b) a step of enrichment of the sample in membrane vesicles, and c) an anion exchange chromatography and/or gel permeation chromatography treatment of the sample.

As indicated above, the sample (e.g. supernatant) enrichment step may comprise one or more centrifugation, clarification, ultrafiltration, nanofiltration and/or affinity chromatography steps on the supernatant. In a first specific embodiment, the enrichment step comprises (i) the elimination of cells and/or cell debris (clarification), possibly followed by (ii) a concentration and/or affinity chromatography step. In an other specific embodiment, the enrichment step comprises an affinity chromatography step, optionally preceded by a step of elimination of cells and/or cell debris (clarification). A preferred enrichment step according to this invention comprises (i) the elimination of cells and/or cell debris (clarification), (ii) a concentration and (iii) an affinity chromatography. The cells and/or cell debris may be eliminated by centrifugation of the sample, for example, at a low speed, preferably below 1000 g, between 100 and 700 g, for example. Preferred centrifugation conditions during this step are approximately 300 g or 600 g for a period between 1 and 15 minutes, for example.

The cells and/or cell debris may also be eliminated by filtration of the sample, possibly combined with the centrifugation described above. The filtration may particularly be performed with successive filtrations using filters with a decreasing porosity. For this purpose, filters with a porosity above 0.2 .mu.m, e.g. between 0.2 and 10 .mu.m, are preferentially used. It is particularly possible to use a succession of filters with a porosity of 10 .mu.m, 1 .mu.m, 0.5 .mu.m followed by 0.22 .mu.m.

A concentration step may also be performed, in order to reduce the volumes of sample to be treated during the chromatography stages. In this way, the concentration may be obtained by centrifugation of the sample at high speeds, e.g. between 10,000 and 100,000 g, to cause the sedimentation of the membrane vesicles. This may consist of a series of differential centrifugations, with the last centrifugation performed at approximately 70,000 g. The membrane vesicles in the pellet obtained may be taken up with a smaller volume and in a suitable buffer for the subsequent steps of the process. The concentration step may also be performed by ultrafiltration. In fact, this ultrafiltration allows both to concentrate the supernatant and perform an initial purification of the vesicles. According to a preferred embodiment, the biological sample (e.g., the supernatant) is subjected to an ultrafiltration, preferably a tangential ultrafiltration. Tangential ultrafiltration consists of concentrating and fractionating a solution between two compartments (filtrate and retentate), separated by membranes of determined cut-off thresholds. The separation is carried out by applying a flow in the retentate compartment and a transmembrane pressure between this compartment and the filtrate compartment. Different systems may be used to perform the ultrafiltration, such as spiral membranes (Millipore, Amicon), flat membranes or hollow fibres (Amicon, Millipore, Sartorius, Pall, GF, Sepracor). Within the scope of the invention, the use of membranes with a cut-off threshold below 1000 kDa, preferably between 300 kDa and 1000 kDa, or even more preferably between 300 kDa and 500 kDa, is advantageous. The affinity chromatography step can be performed in various ways, using different chromatographic support and material. It is advantageously a non-specific affinity chromatography, aimed at retaining (i.e., binding) certain contaminants present within the solution, without retaining the objects of interest (i.e., the exosomes). It is therefore a negative selection. Preferably, an affinity chromatography on a dye is used, allowing the elimination (i.e., the retention) of contaminants such as proteins and enzymes, for instance albumin, kinases, deshydrogenases, clotting factors, interferons, lipoproteins, or also co-factors, etc. More preferably, the support used for this chromatography step is a support as used for the ion exchange chromatography, functionalised with a dye. As specific example, the dye may be selected from Blue SEPHAROSE® (Pharmacia), YELLOW 86, GREEN 5 and BROWN 10 (Sigma). The support is more preferably agarose. It should be understood that any other support and/or dye or reactive group allowing the retention (binding) of contaminants from the treated biological sample can be used in the instant invention.

In one embodiment a membrane vesicle preparation process within the scope of this invention comprises the following steps: a) the culture of a population of membrane vesicle (e.g. exosome) producing cells under conditions enabling the release of vesicles, b) the treatment of the culture supernatant with at least one ultrafiltration or affinity chromatography step, to produce a biological sample enriched with membrane vesicles (e.g. with exosomes), and c) an anion exchange chromatography and/or gel permeation chromatography treatment of the biological sample. In a preferred embodiment, step b) above comprises a filtration of the culture supernatant, followed by an ultrafiltration, preferably tangential. In another preferred embodiment, step b) above comprises a clarification of the culture supernatant, followed by an affinity chromatography on dye, preferably on Blue SEPHAROSE™.

In addition, after step c), the material harvested may, if applicable, be subjected to one or more additional treatment and/or filtration stages d), particularly for sterilisation purposes. For this filtration treatment stage, filters with a diameter less than or equal to 0.3 .mu.m are preferentially used, or even more preferentially, less than or equal to 0.25 .mu.m. Such filters have a diameter of 0.22 .mu.m, for example.

After step d), the material obtained is, for example, distributed into suitable devices such as bottles, tubes, bags, syringes, etc., in a suitable storage medium. The purified vesicles obtained in this way may be stored cold, frozen or used extemporaneously. Therefore, a specific preparation process within the scope of the invention comprises at least the following steps: c) an anion exchange chromatography and/or gel permeation chromatography treatment of the biological sample, and d) a filtration step, particularly sterilising filtration, of the material harvested after stage c). In a first variant, the process according to the invention comprises: c) an anion exchange chromatography treatment of the biological sample, and d) a filtration step, particularly sterilising filtration, on the material harvested after step c).

### Example 1: Amniotic Fluid Stem Cell Exosomes Inhibit U87 Proliferation

Approximately 2 to 5 ml of fresh amniotic fluid was harvested from women undergoing routine amniocentesis at 16 to 21 weeks of pregnancy (2.sup.nd trimester). Second trimester amniotic fluid contained approximately 1-2.times.10.sup.4 live cells per ml. The cells were pelleted in a clinical centrifuge and resuspended in 15 ml multipotent amniotic fluid/fetal stem cells “MAFSC” medium. MAFSC medium was composed of low glucose Dulbecco Modified Eagle's Medium (GIBCO, Carlsbad, Calif.) and MCDB 201 medium (SIGMA, Saint Louis, Mo.) at a one to one ratio and contained 2% Defined Fetal Calf Serum (HYCLONE, Logan, Utah), 1.time. insulin-transferrin-selenium, linoleic-acid-bovine-serum-albumin (ITS+1, SIGMA), 1 nanomolar dexamethasone (Sigma), 100 .mu.m ascorbic acid 2-phosphate (Sigma), 4 .mu.m/ml gentamycin, 10 ng/ml of rhEGF (R&D Systems, Minneapolis, Minn.), 10 ng/ml rrPDGF-BB (R&D) and 10 ng/ml rhFGF-basic (R&D). The wells of 6-well culture dishes were prepared for cell plating by coating for one hour at room temperature with 2.5 ml of fibronectin (stock of 10 .mu.g fibronectin/ml of sterile water) immediately prior to cell plating. The fibronectin solution was removed prior to cell plating and the wells were not washed after removal of the fibronectin solution. The cells were then seeded in 2.5 ml of medium in each well.

The cells in MAFSC culture appeared under the inverted phase microscope as large suspension cells that divided on average once every 4 days, but ceased dividing 8-12 days after seeding. The growth medium of MAFSC cultures was changed with complete MAFSC medium every two days making sure to not lose the suspended cells. After 8-10 days, small numbers of adherent cells emerged which grew into large colonies of >10.sup.5 cells in 14-15 days. On average, 0-1 adherent colonies grew out per 2.times.10.sup.4 live cells seeded. Hence, a sample of 5 ml of fresh amniotic fluid gave rise to 3-5 adherent cell colonies, resulting in a single colony/clone in the majority of the wells of 6-well cell culture clusters.

Cells were transferred to successively larger fibronectin-coated flasks/vessels. To perform cell transfer, the cells were grown to a subconfluent state of approximately 40% confluence and were detached with 0.25% Trypsin-EDTA and replated at a 1:3 or 1:12 dilution under the same culture conditions. MAFSC, or otherwise defined here as amniotic fluid stem cells, were used as a source of exosomes. As a control, fetal calf serum exosomes, and bone marrow mesenchymal stem cell derived exosomes were added to the tissue culture at the indicated ratio. Exosomes were concentrated with ExoQuick (Boston Mass.) according to the manufacturer's instructions. Quantification of exosomes was performed by Bradford Assay.

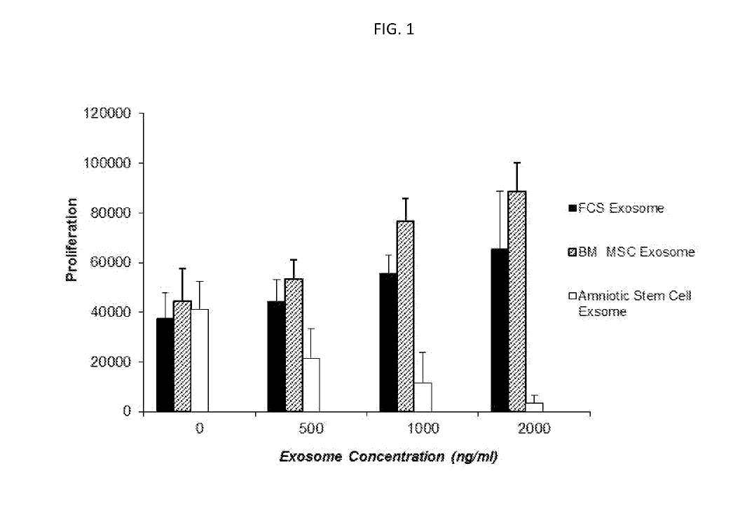

Exosomes where added to U87 MG cells (ATCC) cultured in 96 well flat-bottomed plates for 48 hours at a concentration of 50,000 cells per well in EMEM media with 10% FCS and pen strept mixture. Proliferation was quantified by tritiated thymidine incorporation. 1 microCurie per well of tritiated thymidine was added in the last 8 hours of culture and quantification of incorporated thymidine was performed by scintillation counting. Results are shown in the bar graph of FIG. 1.

1. A method of treating glioma comprising the steps of: a) identifying a patient suffering from glioma; b) obtaining amniotic fluid; c) extracting from said amniotic fluid a population of cells with ability in inhibit neoplastic activity of glioma or other brain neoplasms; d) expanding said amniotic fluid cells with ability in inhibit neoplastic activity of glioma or other brain neoplasms in a manner to allow for increased number of cells while maintaining said ability inhibit neoplastic activity of glioma or other brain neoplasms, optionally treating said cells under conditions resembling the tumor microenvironment; and e) administering said expanded amniotic fluid derived cells into a patient suffering from glioma.

2. The method of claim 1, wherein said amniotic fluid stem cells are treated under conditions of hypoxia.

3. The method of claim 1, wherein said glioma refers to: a) a glioblastoma; b) a glioblastoma multiforme; c) an oligodendroglioma; d) a primitive neuroectodermal tumor; e) an astrocytoma; f) an ependymoma; g) an oligodendroglioma; h) a medulloblastoma; i) a meningioma; j) a pituitary carcinoma; k) a neuroblastoma; or 1) a craniopharyngioma.

4. The method of claim 2, wherein said conditions resembling tumor microenvironment are achieved by culture under hypoxia.

5. The method of claim 1, wherein said conditions resembling tumor microenvironment are achieved by culture in tumor cell line conditioned media.

6. The method of claim 1, wherein said conditions resembling tumor microenvironment are achieved by culture in a combination containing growth factors selected from a group comprising of: a) VEGF; b) TGF-beta; c) PGE-2; and d) IL-10.

7. The method of claim 1, wherein said conditions resembling tumor microenvironment are achieved by culture with tumor derived exosomes.

8. The method of claim 1, wherein said conditions resembling tumor microenvironment are achieved by culture in NGF.

9. The method of claim 1, wherein said conditions resembling tumor microenvironment are achieved by culture in HCG.

10. The method of claim 1, wherein said stimulation of immunogenicity of said endothelial cells is achieved through culture with a histone deacetylase inhibitor.

11. The method of claim 10, wherein said histone deacetylase inhibitor is an epigenetic acting drug.

12. The method of claim 11, wherein said epigenetic acting drug is 5-azacytidine.

13. The method of claim 11, wherein said histone deacetylase inhibitor is selected from a group comprising of: a) trichostatin-A; b) valproic acid; c) sodium phenylbutryate; and d) lithium dichloride.

14. The method of claim 13, wherein said valproic acid is used to culture cells for a period of approximately 48 hours at a concentration of approximately 1 mM valproic acid.
